(19) United States
(12) Patent Application Publication
(10) Pub. No.: US 2009/0143469 A1
(43) Pub. Date: Jun. 4, 2009

(54) SALTS OF NATEGLINIDE

(76) Inventors: Paul Allen Sutton, Parsippany, NJ (US); Richard V. Vivilecchia, Rockaway, NJ (US); David John Parker, West Milford, NJ (US); Marilyn De La Cruz, Matawan, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 12/366,936

(22) Filed: Feb. 6, 2009

Related U.S. Application Data
(63) Continuation of application No. 10/507,255, filed on Sep. 28, 2004, now abandoned.
(60) Provisional application No. 60/363,178, filed on Mar. 11, 2002.

(51) Int. Cl. A61K 31/195; C07C 229/02; A61P 3/00; A61P 9/00

(52) U.S. Cl. 514/563; 562/450

(57) ABSTRACT

The present invention relates to salts of organic acid, in particular salt of nateglinide, combined preparations comprising one or more salts of nateglinide and, optionally, one or more additional ingredients and the use thereof in pharmaceutical compositions for preventing or treating diabetes, cardiovascular disease, or conditions associated therewith.

### FIELD OF THE INVENTION

The present invention relates to salts of nateglinide, combined preparations comprising one or more salts of nateglinide and, optionally, one or more additional ingredients and the use thereof in pharmaceutical compositions for preventing or treating diabetes, cardiovascular diseases, or conditions associated therewith.

### BACKGROUND OF THE INVENTION

### N-(trans-4-isopropylcyclohexylcarbonyl)-D-phenylalanine, also Known as Nateglinide has the Formula (I)

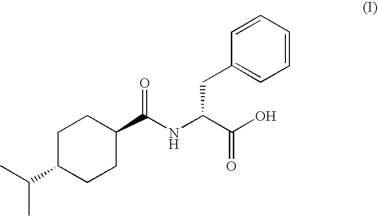

Nateglinide is disclosed in U.S. Pat. No. 4,816,484 and EP 0 196 222 Nateglinide is known to have several crystal forms, such as B-type and H-type crystals. The H-type crystals and methods for their production are described in U.S. Pat. No. 5,463,116 and EP 0 526 171. Compositions containing nateglinide are commercially available, e.g. from Novartis under the trademark STARLIX®. Nateglinide has therapeutic utility in lowering blood glucose levels by stimulating insulin secretion from the pancreas, and thus, has been used in the treatment of diabetes.

There remains a need, however, to improve the solubility of nateglinide in aqueous systems, to increase the absorption of nateglinide, and to increase the stability of nateglinide in galenic formulations.

### SUMMARY OF THE INVENTION

The present invention relates to salts of nateglinide, combined preparations comprising one or more salts of nateglinide and, optionally, one or more additional ingredients and the use thereof in pharmaceutical compositions for preventing or treating diabetes, cardiovascular diseases, or conditions associated therewith. In particular the present invention relates to salts of the anion of nateglinide with an appropriate cation selected from the group consisting of sodium, potassium, calcium, magnesium, ammonium, N-methyl-D-glucamin, tris(hydroxymethyl)-aminomethane and lysine.

According to one aspect, there are salts of nateglinide provided that have a melting point in the range of 50 to 300° C. In one preferred aspect the melting point of the salts of nateglinide according to the invention have a melting point in the range of 150 to 300° C. In another preferred aspect, the salts of nateglinide according to the invention have a melting point in the range of 55 to 125° C.

In another aspect of the invention, there are salts of nateglinide provided that have a solubility in water of at least 0.18 mg/ml. In a more preferred aspect the salts of nateglinide according to the invention have a solubility in water of at least 0.4 mg/ml and in a most preferred aspect the salts of nateglinide according to the invention have a solubility in water of at least 40 mg/ml.

The salts of nateglinide according to the invention have generally a higher degree of dissociation in water, and thus, substantially improved water solubility. In addition, the higher water solubility can, under certain conditions, lead to increased biological availability of the salts, salt hydrates or salt anions in the case of solid dosage forms, which is beneficial to patients. Furthermore, some of the salts according to the invention have proved to be exceptionally physically stable, particularly the alkaline earth salts. For different relative humidities at room temperature and also at slightly higher temperatures, the salts, including salt hydrates, according to the invention show, with the exception of a potassium and a calcium salt, practically no water absorption or water loss over a wide range of humidities and for periods of a few hours, e.g. four hours. Also, for example, the melting point of the salts according to the invention will not be changed by storing under different relative humidities, except for the melting point of those salts that are hygroscopic or moderately hygroscopic.

Another aspect of the present invention relates to a composition comprising one or more salts of nateglinide. In a preferred aspect, such a composition is a combined preparation or pharmaceutical composition. More preferred, such a pharmaceutical composition is for the treatment of diabetes, cardiovascular diseases, or conditions associated therewith.

Still another aspect of the present invention relates to the use of a salt of nateglinide according to the present invention for the manufacture of a drug for the treatment of diabetes, cardiovascular diseases, or conditions associated therewith.

Still another aspect of the present invention relates to a method of treatment of diabetes, cardiovascular diseases, or conditions associated therewith comprising the administration, to a mammal in need of such treatment, of an effective amount of a salt of nateglinide according to the present invention, or a combination or a pharmaceutical composition comprising the same.

As used herein the terms “combination comprising a salt of nateglinide” and “pharmaceutical composition comprising a salt of nateglinide” are meant to include such combinations or pharmaceutical compositions that contain more than one salt of nateglinide, e.g. two different salts of nateglinide.

Further, the invention relates to a method of preparing a salt of nateglinide by treating a solution of nateglinide with a suitable base reactant.

Also, an aspect of the present invention concerns a method of preparation of a salt of nateglinide comprising the addition of a solution of a salt of calcium or a salt of magnesium in a suitable solvent to a solution of the sodium or potassium salt of nateglinide.

### DETAILED DESCRIPTION OF THE INVENTION

The so called H-type form of nateglinide employed in the reactions to produce the salts of nateglinide according to the invention has a melting point of 140° C., as determined by Differential Thermal Analysis (DTA), and can be prepared according to methods known to those of the art, which were also previously disclosed, e.g. in EP 0 526 171.

The salts of nateglinide according to the present invention include crystalline, semi-crystalline, and amorphous salts of nateglinide. The term “semi-crystalline”, as used herein, shall expressly include mixtures with varying proportions of amorphous and crystalline portions of the salts of nateglinide according to the present invention, respectively. Included within the terms “salt of nateglinide” or “salts of nateglinide” as used herein are solvates formed of pharmaceutically acceptable solvents, such as hydrates, and polymorphous forms of the nateglinide salts. Solvates and especially hydrates of the nateglinide salts may be present, for example, as hemi-, mono-, sesqui-, di-, tri-, tetra-, penta-, hexa-solvates or hydrates, respectively. Solvents used for crystallisation, such as alcohols, especially ethanol, ketones, especially acetone, esters, e.g. ethyl acetate, may be embedded in the crystal grating.

In one embodiment of the present invention, the salt of nateglinide is a sodium salt of nateglinide. The sodium salt is prepared in four different hydrate forms, the hemihydrate, hydrate, sesquihydrate and trihydrate. All of these forms are crystalline. The sodium salts are very advantageous in view of their water solubility. The water solubility of the sodium salts of nateglinide is in excess of 40 mg/ml. This may provide for a greater and also faster bioavailability of the substance, in particular in combined preparations or pharmaceutical compositions that contain a combination of nateglinide and one or more salts thereof with different solubilities to establish a formulation with a desired profile of efficacy or action.

In another embodiment of the present invention, the salt of nateglinide is a potassium salt of nateglinide. Four different salt forms, one anhydrous form and three hydrate forms of the potassium salt of nateglinide have been prepared and characterised. One of the hydrate forms is very hygroscopic and forms a dihydrate in an atmosphere of 84% relative humidity. The potassium salts according to the present invention are also very favourable because of their high water solubility of more than 40 mg/ml.

In another embodiment of the present invention, the salt of nateglinide is a calcium salt of nateglinide. The present inventors have prepared two polymorphic hydrate forms of the calcium salt of nateglinide, one of which is only slightly hygroscopic, whereas the other is not hygroscopic at all. The bulk density of the calcium salts is higher and, therefore, improved over e.g. the sodium salts. The water solubility of the calcium salts of nateglinide is much higher than that of the free acid of nateglinide.

A magnesium salt of nateglinide has also been prepared. The prepared salt crystallises as a non-hygroscopic mono-hydrate with a favourable bulk density and a water solubility comparable to that of the calcium analogue.

In still another embodiment of the present invention, the salt of nateglinide is an ammonium salt of nateglinide. This salt crystallizes into various anhydrous forms.

In a further embodiment of the present invention, the salt of nateglinide is the N-methyl-D-glucamine salt of nateglinide. The N-methyl-D-glucamine salt of nateglinide is a non-hygroscopic, anhydrous material with a bulk density comparable to that of the alkaline metal salts of nateglinide. The water solubility of this salt is lower than that of the alkaline metal salts of nateglinide but still conspicuously higher than that of the alkaline earth metal salts of nateglinide.

In still another embodiment of the present invention, the salt of nateglinide is the tris(hydroxymethyl)-aminomethane salt of nateglinide. This salt exists as well defined rods. However, it is presently not clear whether this salt is a hemihydrate or a dihydrate. On dehydration the salt became amorphous.

Surprisingly, the bulk density of this salt is also relatively high. It is about the same as that of the lysine salt of nateglinide and, thus, presents a considerable improvement over the bulk density of the free acid, for instance. The tris(hydroxymethyl)-aminomethane salt of nateglinide also has a high solubility in water of more than 40 mg/ml.

In still another embodiment of the present invention, the salt of nateglinide is the lysine salt of nateglinide. Three different forms of this salt, one anhydrous form, a sesquihydrate and a dihydrate have been prepared. The sesquihydrate was found to be moderately hygroscopic. Further it was very unexpected to find that the bulk density of the lysine salt of nateglinide was markedly improved with respect to the free acid and the other salts of nateglinide disclosed hereinabove. The water solubility of the lysine salt is comparable to that of the N-methyl-D-glucamine salt of nateglinide and, therefore, still considerably higher than that of the free acid.

The salts of nateglinide are prepared by forming a solution of nateglinide in a solvent in which nateglinide is soluble at an ambient temperature, and adding a solution of the base reactant in the same or a different solvent. Optionally, cooling the solution or adding another solvent, e.g. with a lower solubility for the resultant salt of nateglinide, can enhance the precipitation of the salts of nateglinide. The precipitated salts of nateglinide are then isolated, e.g. by filtration, and dried.

Examples of solvents, preferably pharmaceutically acceptable solvents, are acetonitrile, esters such as methyl acetate, ethyl acetate and water, as well as toluene, and the like. Acetonitrile and ethyl acetate are particularly effective. Preferred mixed solvents include a mixture of a polar solvent such as acetonitrile, acetone and a lower alcohol, such as ethanol and isopropanol, with water. The ambient temperature, i.e., the temperature of dissolution, ranges preferably from room temperature to about the boiling point of the solvent, and more preferably from room temperature to 80° C. The amount of nateglinide in the solvent ranges preferably from 1 to 50% by weight of the resulting mixture. On the other hand, it is not efficient in terms of the volume of the solvent required to use less than 1% of nateglinide by weight. The lower temperature to which the prepared solution of nateglinide salt can be cooled to induce or promote precipitation of the desired crystal form of nateglinide ranges preferably from room temperature to about −15° C., and more preferably from about 5 to about 0° C. It may be advantageous to add seed crystals to the solution to further aid precipitation. The resulting mixture way then be maintained at the lower temperature for a time sufficient to assure complete precipitation of the desired form of the salt of nateglinide.

In one embodiment of the invention, the calcium or the magnesium salt is precipitated from a solution of the sodium salt of nateglinide upon addition of a solution of calcium chloride or magnesium chloride, respectively.

Regarding the shape of the crystalline salts, those shapes that lead to a higher bulk density of the resultant salt of nateglinide are generally preferred. Thus, a rod shape is, for instance, preferred over a needle shape, since it was determined that needles have poorer bulk density than rods.

The salts of nateglinide of the invention preferably exist in essentially pure form, for example in a degree of purity of >95%, more preferably >98%, and most preferably >99%.

The salts of nateglinide according to the invention are preferably administered in the form of a combined preparation or a pharmaceutical composition comprising additional ingredients. Additional ingredients include natural and/or artificial ingredients, which are commonly used to prepare pharmaceutical compositions. Such ingredients are known to those skilled in the art. Preferably the additional ingredients are used in the compositions of the invention in an amount that corresponds to an amount generally recognized as both safe and effective by the United States Food and Drug Administration, the Environmental Protection Agency, the United States Department of Agriculture, or other comparable regulatory agency. For those additional ingredients for which no regulatory approval has been obtained, then an amount generally accepted in the art as both safe and efficacious is preferred.

Further, one or more salts of nateglinide according to the present inventions are administered in the form of a combined preparation or pharmaceutical composition as described hereinbefore that comprises one or more additional pharmaceutically active substances.

In a particularly preferred embodiment the additional pharmaceutically active substance is an antidiabetic. It is further preferred that this ingredient is an insulin secretion enhancer or an insulin sensitizer. In an alternative embodiment the at least one further active ingredient is selected from the group consisting of substances used in the treatment of non-diabetic conditions.

In another particularly preferred embodiment the additional pharmaceutically active substance is a rennin inhibitor, an ACE inhibitor or an angiotensin II inhibitor, the latter also being named as ATₐ-receptor antagonist.

The term “antidiabetic” generally comprises the compounds, substances and compositions known to those of ordinary skill to be used in the treatment of type 1 and type 2 diabetes mellitus. This term in particular comprises insulin secretion enhancers and insulin sensitizers, as well as dipeptidyl peptidase IV (DPP IV) antagonists.

Insulin secretion enhancers are pharmacological active compounds having the property to promote secretion of insulin from pancreatic β-cells. Examples for insulin secretion enhancers include nateglinide, repaglinide, glucagon receptor antagonists, sulphonyl urea derivatives, incretin hormones, especially glucagon-like peptide-1 (GLP-1) or GLP-1 agonists, β-cell imidazoline receptor antagonists, and BTS 67582 described by T. Page et al in Br. J. Pharmacol. 1997, 122, 1464-1468.

Repaglinde can be administered in the form as it is marketed e.g. under the trademark NovoNorm™.

The term “glucagon receptor antagonists” as used herein relates in particular to the compounds described in WO 98/04528, especially BAY27-9955, and those described in Bioorg Med. Chem. Lett 1992, 2, 915-918, especially CP-99,711, J. Med. Chem. 1998, 41, 5150-5157, especially NNC 92-1687, and J. Biol. Chem. 1999, 274; 8694-8697, especially L-168,049 and compounds disclosed in U.S. Pat. No. 5,880,139, WO 99/01423, U.S. Pat. No. 5,776,954, WO 98/22109, WO 98/22108, WO 98/21957 and WO 97/16442.

The sulphonyl urea derivative is, for example, glisoxepid, glyburide, glibenclamide, acetohexamide, chlorpropamide, glibornuride, tolbutamide, tolazamide, glipixide, carbutamide, gliquidone, glyhexamide, phenbutamide or tolcyclamide; and preferably glimepiride or gliclazide. Tolbutamide, glibenclamide, gliclazide, glibornuride, gliquidone, glisoxepid and glimepiride can be administered e.g. in the form as they are marketed under the trademarks RASTINON HOECHST™, AZUGLUCON™, DIAMICRON™, GLUBORID™, GLURENORM™, PRO-DIABAN™ and AMARYL™, respectively.

GLP-1 is a insulinotropic proteine which was described, e.g., by W. E. Schmidt et al. in Diabetologia 28, 1985, 704-707 and in U.S. Pat. No. 5,705,483. The term “GLP-1 agonists” used herein means variants and analogs of GLP-1(7-36)NH₂ which are disclosed in particular in U.S. Pat. No. 5,120,712, U.S. Pat. No. 5,118,666, U.S. Pat. No. 5,512,549, WO 91/11457 and by C. Orskov et al in J. Biol. Chem. 264 (1989) 12826.

The term “GLP-1 agonists” comprises especially compounds like GYP-1(7-37), in which compound the carboxy-terminal amide functionality of Arg³⁶ is displaced with Gly at the 37^{th} position of the GLP-1(7-36)NH₂ molecule and variants and analogs thereof including GLN⁹-GLP-1(7-37), D-GLN⁹-GLP-1(7-37), acetyl LYS⁹-GLP-1(7-37), LYS¹⁸-GLP-1(7-37) and, in particular, GLP-1(7-37)OH, VAL⁸-GLP-1(7-37), GLY⁸-GLP-1(7-37), THR⁸-GLP-1(7-37), MET⁸-GLP-1(7-37) and 4-imidazopropionyl-GLP-1. Special preference is also given to the GLP agonist analog exendin-4, described by Greig et al in Diabetologia 1999, 42, 45-50.

The term “β-cell imidazoline receptor antagonists” as used herein means compounds as those described in WO 00/78726 and by Wang et al in J. Pharmacol. Exp. Ther. 1996; 278; 82-89, e.g. PMS 812.

The term “insulin sensitizer” used herein means any and all pharmacological active compounds that enhance the tissue sensitivity towards insulin. Insulin sensitivity enhancers include, e.g., inhibitors of GSK-3, retinoid X receptor (RXR) agonists, agonists of Beta-3 AR, agonists of UCPS, antidiabetic thiazolidinediones (glitazones), non-glitazone type PPAR-T agonists, dual PPARγ/PPARα agonists, antidiabetic vanadium containing compounds and biguanides, e.g., metformin.

The insulin sensitivity enhancer is preferably selected from the group consisting of antidiabetic thiazolidinediones, antidiabetic vanadium containing compounds and metformin.

Examples of “inhibitors of GSK-3” include, but are not limited to those disclosed in WO 00/21927 and WO 97/41854.

By “RXR agonist” is meant a compound or composition which when combined with RXR homodimers or heterodimers increases the transcriptional regulation activity of RXR, as measured by an assay known to one skilled in the art, including, but not limited to, the “co-transfection” or “cis-trans” assays described or disclosed in U.S. Pat. Nos. 4,981,784, 5,071,773, 5,298,429, 5,506,102, WO89/05355, WO91/06677, WO92/05447, WO93/11235, WO95/18380, PCT/US93/04399, PCT/US94/03795 and CA 2,034,220, which are incorporated by reference herein. It includes, but is not limited to, compounds that preferentially activate RXR over RAR (i.e. RXR specific agonists), and compounds that activate both RXR and RAR (i.e. pan agonists). It also includes compounds that activate RXR in a certain cellular context but not others (i.e. partial agonists). Compounds disclosed or described in the following articles, patents and patent applications which have RXR agonist activity are incorporated by reference herein: U.S. Pat. Nos. 5,399,586 and 5,466,861, WO96/05165, PCT/US95/16842, PCT/US95/16695, PCT/US93/10094, WO94/15901, PCT/US92/11214, WO93/11755, PCT/US93/10166, PCT/US93/10204, WO94/15902, PCT/US93/03944, WO93/21146, provisional applications 60,004,897 and 60,009,884, Boehm, et al. J. Med. Chem. 38(16):3146-3155, 1994, Boehm, et al. J. Med. Chem. 37(18); 2930-2941, 1994, Antras et al., J. Biol. Chem. 266:1157-1161 (1991), Salazar-Olivo et al., Biochem. Biophys. Res. Commun. 204:157-263 (1994) and Safanova, Mol. Cell. Endocrin. 104:201-211 (1994). RXR specific agonists include, but are not limited to, LG 100268 (i.e. 2-[1-(3,5,5,8,8-pentamethyl-5,6,7,8-tetrahydro-2-naphthyl)-cyclopropyl]-pyridine-5-carboxylic acid) and LGD 1069 (i.e. 4-[(3,5,5,8,8-pentamethyl-5,6,7,8-tetrahydro-2-naphthyl)-2-carbonyl]-benzoic acid), and analogs, derivatives and pharmaceutically acceptable salts thereof. The structures and syntheses of LG 100268 and LGD 1069 are disclosed in Boehm et al. J. Med. Chem. 38(16):3146-3155, 1994, incorporated by reference herein. Pan agonists include, but are not limited to, ALRT 1057 (i.e. 9-cis retinoic acid), and analogs, derivatives and pharmaceutically acceptable salts thereof.

Examples of “agonists of Beta-3 AR” include, bur are not limited to CL-316,243 (Lederle. Laboratories) and those disclosed in WO 99/29672, WO 98/32753, WO 98/20005, WO 98/09625, WO 97/46556, WO 97/37646 and U.S. Pat. No. 5,705,515.

The term “agonists of UCPs” used herein means agonists of UCP-1, preferably UCP-2 and even more preferably UCP-3. UCPs are disclosed in Vidal-Puig et al., Biochem. Biophys Res. Commun., Vol. 235(1) pp. 79-82 (1997). Such agonists are a compound or composition which increases the activity of UCPs.

The antidiabetic thiazolidinedione (glitazone) is, for example, (S)-((3,4-dihydro-2-(phenyl methyl)-2H-1-benzopyran-6-yl)methyl-thiazolidine-2,4-dione (englitazone), S-[[4-(3-(5-methyl-2-phenyl-4-oxazolyl)-1-oxopropyl)-phenyl]-methyl]-thiazolidine-2,4-dione (darglitazone), 5-[[4-(1-methyl-cyclohexyl)methoxy)-phenyl]methyl]-thiazolidine-2,4-dione (ciglitazone), 5-[[4-(2-(1-indolyl)ethoxy)phenyl]methyl]-thiazolidine-2,4-dione (DRF2189), 5-{4-[2-(5-methyl-2-phenyl-4-oxazolyl)-ethoxy)]benzyl}-thiazolidine-2,4-dione (BM-13.1246), 5-(2-naphthylsulfonyl)-thiazolidine-2,4-dione (AY-31637), bis[4-[(2,4-dioxo-5-thiazolidinyl)-methyl]phenyl)methane (YM268), 5-(4-[2-(5-methyl-2-phenyl-4-oxazolyl)-2-hydroxyethoxy]-benzyl]-thiazolidine-2,4-dione (AD-5075), 5-[[4-(1-phenyl-1-cyclopropanecarbonylamino)-benzyl]-thiazolidine-2,4-dione (DN-108) 5-[(4-(2-(2,3-dihydroindol-1-yl)ethoxy)phenyl-methyl)-thiazolidine-2,4-dione, 5-[3-(4-chloro-phenyl])-2-propynyl]-5-phenylsulfonyl)thiazolidine-2,4-dione, 5-[3-(4-chlorophenyl])-2-propynyl]-5-(4-fluorophenyl-sulfonyl)thiazolidine-2,4-dione, 5-[[4-(2-(methyl-2-pyridinyl-amino)-ethoxy)phenyl]methyl]-thiazolidine-2,4-dione (rosiglitazone), 5-{[4-(2-(5-ethyl-2-pyridyl)ethoxy)phenyl]-methyl}thiazolidine-2,4-dione (pioglitazone), 5-[[4-((3,4-dihydro-6-hydroxy-2,5,7,8-tetramethyl-2H-1-benzopyran-2-yl)methoxy)-phenyl]-methyl]-thiazolidine-2,4-dione (troglitazone), 5-[6-(2-fluoro-benzyloxy)naphthalen-2-ylmethyl]-thiazolidine-2,4-dione (MCC555), 5-[[2-(2-naphthyl)-benzoxazol-5-yl]-methyl]thiazolidine-2,4-dione (T-174) and 5-(2,4-dioxothiazolidin-5-ylmethyl)-2-methoxy-N-(4-trifluoromethyl-benzyl)benzamide (KRP297).

More preferably, the thiazolidinedione is selected from the group consisting of 5-[[4-(2-(methyl-2-pyridinyl-amino)ethoxy)phenyl]methyl]-thiazolidine-2,4-dione (rosiglitazone), 5-{[4-(2-(5-ethyl-2-pyridyl)ethoxy)phenyl]-methyl}thiazolidine-2,4-dione (pioglitazone) and 5-{[4-((3,4-dihydro-6-hydroxy-2,5,7,8-tetramethyl-2H-1-benzopyran-2-ylmethoxy)-phenyl]-methyl}-thiazolidine-2,4-dione (troglitazone), MCC555, T-174 and KRP297, especially rosiglitazone, pioglitazone and troglitazone, or a pharmaceutically acceptable salt thereof.

The glitazones 5-{[4-(2-(5-ethyl-2-pyridyl)ethoxy)phenyl]-methyl}thiazolidine-2,4-dione (pioglitazone, EP 0 193 256 A1), 5-{[4-(2-(methyl-2-pyridinyl-amino)-ethoxy)phenyl]methyl}-thiazolidine-2,4-dione (rosiglitazone, EP 0 306 228 A1), 5-[[4-((3,4-dihydro-6-hydroxy-2,5,7,8-tetramethyl-2H-1-benzopyran-2-yl)methoxy)-phenyl]-methyl]thiazolidine-2,4-dione (troglitazone, EP 0 139 421), (S)-((3,4-dihydro-2-(phenyl-methyl)-2H-1-benzopyran-6-yl)methyl-thiazolidine-2,4-dione (englitazone, EP 0 207 605 B1), 5-(2,4-dioxothiazolidin-5-ylmethyl)-2-methoxy-N-(4-trifluoromethyl-benzyl)benzamide (KRP297, JP 10087641-A), 5-[6-(2-fluoro-benzyloxy)naphthalen-2-ylmethyl]thiazolidine-2,4-dione (MCC555, EP 0 604 983 B1), 5-[{4-(3-(5-methyl-2-phenyl-4-oxazolyl)-1-oxopropyl)-phenyl]-methyl}-thiazolidine-2,4-dione (darglitazone, EP 0 332 332), 5-(2-naphthylsulfonyl)-thiazolidine-2,4-dione (AY-31637, U.S. Pat. No. 4,997,948), 5-{[4-(1-methyl-cyclohexyl)methoxy)-phenyl]methyl}-thiazolidine-2,4-dione (ciglitazone, U.S. Pat. No. 4,287,200) are in each case generically and specifically disclosed in the documents cited in brackets beyond each substance, in each case in particular in the compound claims and the final products of the working examples, the subject-matter of the final products, the pharmaceutical preparations and the claims are hereby incorporated into the present application by reference to these publications. The preparation of DRF2189 and of 5-[[4-(2-(2,3-dihydroindol-1-ylethoxy)phenyl]methyl]-thiazolidine-2,4-dione is described in B. B. Lohray et al., J. Med. Chem. 1998, 41, 1619-1630; Examples 2d and 3g on pages 1627 and 1628. The preparation of 5-[3-(4-chlorophenyl])-2-propynyl]-5-phenylsulfonyl)-thiazolidine-2,4-dione and the other compounds in which A is phenylethynyl mentioned herein can be carried out according to the methods described in J. Wrobel et al. J. Med. Chem. 1998, 41, 1084-1091.

In particular, MCC555 can be formulated as disclosed on page 49, lines 30 to 45, of EP 0 604 983 B1; englitazone as disclosed from page 6, line 52, to page 7, line 6, or analogous to Examples 27 or 28 on page 24 of EP 0 207 605 B1; and darglitazone and 5-{4-[2-(S-methyl-2-phenyl-4-oxazolyl)-ethoxy)]benzyl}-thiazolidine-2,4-dione (BM-13.1246) can be formulated as disclosed on page 8, line 42 to line 54 of EP 0 332 332 B1. AY-31637 can be administered as disclosed in column 4, lines 32 to 51 of U.S. Pat. No. 4,997,948 and rosiglitazone as disclosed on page 9, lines 32 to 40 of EP 0 306 228 A1, the latter preferably as its maleate salt. Rosiglitazone can be administered in the form as it is marketed e.g. under the trademark AVANDIA™. Troglitazone can be administered in the form as it is marketed e.g. under the trademarks ReZulin™, PRELAY™, ROMOZIN™ (in the United Kingdom) or NOSCAL™ (in Japan). Pioglitazone can be administered as disclosed in Example 2 of EP 0 193 256 A1, preferably in the form of the monohydrochloride salt. Corresponding to the needs of the single patient it can be possible to administer pioglitazone in the form as it is marketed e.g. under the trademark ACTOS™. Ciglitazone can, for example, be formulated as disclosed in Example 13 of U.S. Pat. No. 4,287,200.

Non-glitazone type PPARγ agonists are especially N-(2-benzoylphenyl)-L-tyrosine analogues, e.g. GI-262570, and JTT501.

The term “dual PPARγ/PPARα agonists” as used herein means compounds which are at the same time PPARγ and PPARα agonists. Preferred dual PPARγ/PPARα agonists are especially those ω-[(oxoquinazolinylalkoxy)phenyl]alkanoates and analogs thereof, very especially the compound of formula (II)

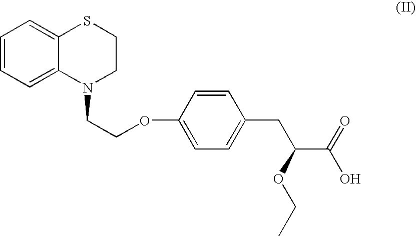

which is described in WO 99/20614 and the compound NC-2100 described by Fukui in Diabetes 2000, 49(5), 759-767.

Preferably, the antidiabetic vanadium containing compound is a physiologically tolerable vanadium complex of a bidentate monoprotic chelant, wherein said chelant is an α-hydroxypyrone or α-hydroxypyridinone, especially those disclosed in the Examples of U.S. Pat. No. 5,866,563, of which the working examples are hereby incorporated by reference, or a pharmaceutically acceptable salt thereof.

In a more preferred embodiment, the insulin sensitivity enhancer is metformin.

The preparation of metformin (dimethyldiguanide) and its hydrochloride salt is state of the art and was disclosed first by Emil A. Werner and James Bell, J. Chem. Soc. 121, 1922, 1790-1794. Metformin, can be administered e.g. in the form as marketed under the trademark GLUCOPHAGE™. The metformin may be present in free form or in the form of a pharmaceutically acceptable salt and includes corresponding stereoisomers as well as the corresponding crystal modifications, e.g. solvates and polymorphs. Preferably, the metformin is metformin hydrochloride.

The term “dipeptidyl peptidase IV antagonists” or “DPP IV antagonists” comprises all activity reducing effectors of the enzyme dipeptidyl peptidase IV as defined and specifically named in WO 97/40832, e.g. isoleucyl-thiazolidid, and also the compounds of the following formulae (III) and (IV)

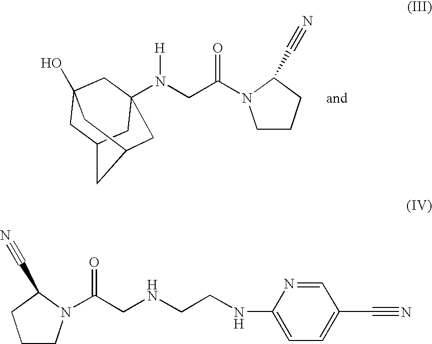

or a pharmaceutically acceptable salt of these compounds, in particular the dihydrochloride of compound of formula (IV). The compound of formula (III) and its preparation is disclosed in WO 00/34241 whereas the compound of formula (IV), its dihydrochloride and its preparation is disclosed in WO 98/19998, the contents of which are hereby incorporated by reference.

The rennin inhibitor preferably employed in the present invention is the compound of formula (V)

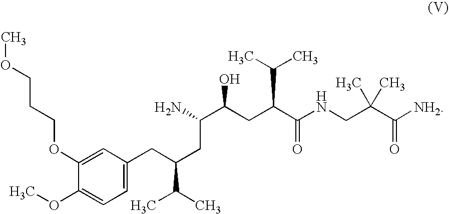

or a pharmaceutically acceptable salt thereof. The renin inhibitor of formula (I), chemically defined as 2(S),4(S),5(S),7(S)—N-(3-amino-2,2-dimethyl-3-oxopropyl)-2,7-di(1-methylethyl)-4-hydroxy-5-amino-8-[4-methoxy-3-(3-methoxy-propoxy)phenyl]-octanamide, is specifically disclosed in EP 678503A. Particularly preferred is the hemi-fumarate salt thereof.

The class of ACE inhibitors comprises compounds having differing structural features. For example, mention may be made of the compounds which are selected from the group consisting alacepril, benazepril, benazeprilat, captopril, ceronapril, cilazapril, delapril, enalapril, enaprilat, fosinopril, imidapril, lisinopril, moveltopril, perindopril, quinapril, ramipril, spirapril, temocapril, and trandolapril, or, in each case, a pharmaceutically acceptable salt thereof.

Preferred ACE inhibitors are those agents that have been marketed, most preferred are benazepril and enalapril.

AT₁-receptor antagonists (also called angiotensin H receptor antagonists or angiotensin hinhibitors) are understood to be those active ingredients that bind to the AT₁-receptor subtype of angiotensin H receptor but do not result in activation of the receptor. As a consequence of the inhibition of the AT₁ receptor, these antagonists can, for example, be employed as antihypertensives or for treating congestive heart failure.

The class of AT₁ receptor antagonists comprises compounds having differing structural features, essentially preferred are the non-peptidic ones. For example, mention may be made of the compounds that are selected from the group consisting of valsartan (cf. EP 443983), losartan (cf. EP253310), candesartan (cf. 459136), eprosartan (cf. EP 403159), irbesartan (cf. EP454511), olmesartan (cf. EP 503785), tasosartan (cf. EP539086), telmisartan (cf. EP 522314), the compound with the designation E-1477 of the following formula (VI)

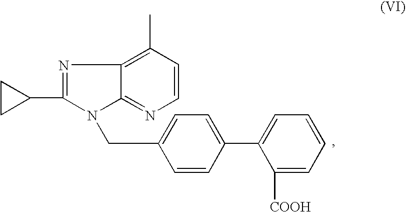

the compound with the designation SC-52458 of the following formula (VII).

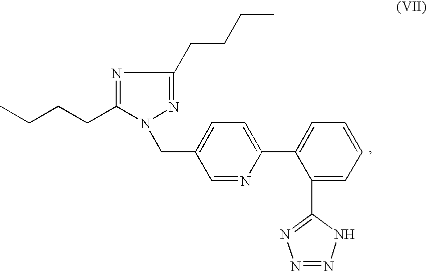

and the compound with the designation the compound ZD-8731 of the following formula (VII)

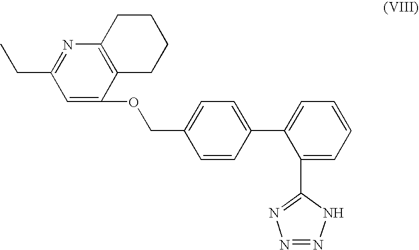

or, in each case, a pharmaceutically acceptable salt thereof.

Preferred AT₁-receptor antagonist are those agents that have been marketed, most preferred is valsartan or a pharmaceutically acceptable salt thereof.

A pharmaceutical preparation which comprises one or more salts of nateglinide and at least one other ingredient and optionally at least one, i.e., one or more, e.g. two, pharmaceutically acceptable carrier for simultaneous, separate or sequential use is especially a “kit of parts” in the sense that the salt of nateglinide or a combination of salts of nateglinide and one or more further pharmaceutically active ingredients, can be dosed independently or by use of different fixed combinations with distinguished amounts of the components, i.e. at different time points or simultaneously. The parts of the kit of parts can then, e.g., be administered simultaneously or chronologically staggered, that is at different time points and with equal or different time intervals for any part of the kit of parts. Preferably, the time intervals are chosen such that the effect on the treated disease or condition in the combined use of the parts is larger than the effect that would be obtained by use of only any one of the components. Preferably, there is at least one beneficial effect, e.g. a mutual enhancing of the effect of one or more salts of nateglinide and at least one further pharmaceutically active ingredient, additional advantageous effects, less side effects, a combined therapeutically effect in an otherwise, e.g. in a monotherapy, non-effective dosage of one or each of the components, and especially a synergism, e.g. a more than additive effect, between said substances and compounds as disclosed herein for combination.

The invention also relates to a commercial package comprising the salt of nateglinide according to the invention, optionally in combination with one or more different salts of nateglinide or other compounds or substances as mentioned hereinbefore, together with instructions for simultaneous, separate or sequential use.

It can be shown by established test models and especially those test models described herein that according to the present invention the combination of one or more salts of nateglinide and, optionally, at least one or more pharmaceutically active ingredients selected from the group comprising nateglinide, repaglinide, metformin, sulfonylureas, thiazolidinedione derivatives, or in each case a pharmaceutically acceptable salt thereof, or at least one of the other compounds disclosed hereinbefore for combination results in a more effective treatment of diseases and conditions mentioned hereinbefore.

Additional benefits resulting from combined treatment are a surprising prolongation of efficacy, a broader variety of therapeutic treatment and reduction of side effects.

Also, for a human patient it is more convenient and easier to remember to take two tablets at the same time, e.g. before a meal, than staggered in time, i.e. according to a more complicated treatment schedule. More preferably, the active ingredients are administered as a fixed combination, i.e. as a single tablet, in all cases described herein. Taking a single tablet is even easier to handle than taking two or more tablets at the same time. Furthermore, the packaging can be accomplished with less effort.

This generally allows the administration of pharmaceutical compositions with relatively small amounts of at least one further active ingredient, in comparison to the amount administered of said ingredient when it is administered alone. Nevertheless it may also be desired to employ said at least one pharmaceutically active ingredient in an amount as if said ingredient was administered alone to considerably enhance it's effect.

It is, however, generally preferred to use as little as possible of said at least one additional pharmaceutically active ingredient, i.e. an amount that, in combination with one or more salts of nateglinide, elicits the desired therapeutic effect. This leads to the advantage that possible side effects of said at least one further active ingredient are kept on a minimum and, thus, in an at least more tolerable range. On the other hand it is also possible to enhance the efficacy of said at least one further active ingredient and thereby shorten the period required for successful treatment.

The person skilled in the pertinent art is fully enabled to select a relevant animal test model to prove the hereinbefore and hereinafter indicated therapeutic indications and beneficial effects. The pharmacological activity may, for example, be demonstrated following essentially an in-vivo test procedure in mice or in a clinical study as described hereinafter.

### In-Vivo Test in Mice for Blood Glucose Control

ICR-CDI mice (female, five weeks old, body weight: about 20 g) are abstained from food for 18 hours, and then used as test subjects. The composition, e.g. combined preparation or pharmaceutical composition, according to the present invention is suspended in 0.5% CMC-0.14M sodium chloride buffer solution (pH 7.4) or suspended in 0.5 percent by weight. The solution or suspension thus obtained is administered orally in fixed volume amounts to the test subjects. After predetermined time, the percentage decrease of the blood glucose against the control group is determined.

### In-Vivo Test for HbA_{1c}

For example, the following procedure can be followed in order to take blood samples: The subject is advised not to take the morning dose of study medication or cat breakfast on the day of a scheduled study visit. The morning dose is administered by site personnel after the collection of all fasting laboratory samples and completion of all study procedures. Visits are scheduled to be performed at 2 week intervals during Period I, and 4 to 8 week intervals during Period II. Subjects have fasted for at least 7 hours at the time of each visit. All blood samples for laboratory evaluations are drawn between 7:00 AM and 10:00 AM. All tests are conducted in accordance with GLP (Good Laboratory Practice) principles following procedures known in the art.

HbA_{1c} is measured by High Performance Liquid Chromatography (HPLC) using the ion-exchange method on a Bio-Rad Diamat analyzer. A back-up affinity method is used if hemoglobin variants or hemoglobin degradation peaks are observed.

Further parameters to be determined are fasting plasma glucose (FPG), fasting lipids (total, HDL (high density lipoprotein)- and LDL (low density lipoprotein)-cholesterol, and triglycerides) and body weight. FPG will be measured using the hexokinase method and LDL-cholesterol will be calculated using the Friedewald formula if triglycerides are <400 mg/dL (4.5 mmol/l).

Hematocrit and hemogloblin, platelet count, erythrocyte count, total and differential leukocyte count (basophils, eosinophils, lymphocytes, monocytes, segmented neutrophils and total neutrophils); albumin, alkaline phosphatase, alanine amino transferase (serum glutamic pyruvic transaminase), aspartate amino transferase (serum glutamic oxalacetic transaminase), blood urea nitrogen or urea, bicarbonate, calcium, chloride, total creatine phosphokinase (CPK), creatine phosphokinase muscle-brain fraction isoenzyme (if CPK is elevated), direct bilirubin, creatinine, γ-glutamyl transferase, lactate dehydrogenase, potassium, sodium, total bilirubin, total protein and uric acid in the blood; and bilirubin, glucose, ketones, pH, protein, and specific gravity in the subjects urine is determined by laboratory analysis. Furthermore, body weight, blood pressure (systolic and diastolic, after 3 minutes sitting) and radial pulse (after 3 minutes sitting) are determined during the visit.

The results clearly show that the salts of nateglinide according to the present invention can be used for the treatment of metabolic disorders and in particular for diabetes, cardiovascular disorders, or conditions associated therewith.

The administration of one or more salts of nateglinide and, optionally, at least one further pharmaceutically active ingredient selected from the group comprising nateglinide, repaglinide, metformin, sulfonylureas, and thiazolidinedione results in a beneficial and more than additional, especially a synergistic or potentiating, therapeutic effect, especially on type 2 diabetes, and also in additional benefits such as a surprising prolongation of efficacy of the drug, a broader variety of therapeutic treatment, providing a good initial blood glucose control in patients, only modest changes in fasting plasma glucose level, and further surprising beneficial effects, comprising e.g. loss of body weight, a decrease of gastrointestinal side effects or an improved safety profile, compared to a monotherapy applying only one of the pharmaceutically active compounds used in the combinations disclosed herein. In particular, the further surprising beneficial effects can be observed during the treatment of diabetes, cardiovascular diseases and during the treatment of conditions associated therewith. Further benefits are that lower doses of the individual drugs to be combined according to the present invention can be used to reduce the dosage, for example, that the dosages need not only often be smaller but are also applied less frequently, or can be used in order to diminish the incidence of side effects (e.g. anaemia, oedema, headache).

Furthermore, in a number of combinations as disclosed herein the side-effects observed with one of the components surprisingly do not accumulate on application of the combination.

The beneficial therapeutic effect, additional benefits and especially the surprising beneficial effects are observed in particular with nateglinide. Very good results have been obtained with the combination of a salt of nateglinide and metformin or metformin hydrochloride.

With the above test models it is also possible to assess the applicability of the inventive combined preparation or pharmaceutical composition with respect to diabetes, cardiovascular diseases, or conditions associated therewith.

Further agents can be employed in forming combined, preparations or pharmaceutical compositions as set forth hereinabove as is well known in the art.

The total amount of additional ingredients in the pharmaceutical compositions according to the invention are preferably from about 30 to about 75 weight percent, based on the total weight of the composition. More preferably, the total amount of additional ingredients is from about 50 to about 70 weight percent, most preferably from about 53 to about 67 weight percent, based on the total weight of the pharmaceutical composition.

The pharmaceutical compositions according to the invention may be in the form of powder, granules, solution, suspension, emulsion, capsule, cachet, tablet and combinations thereof. The compositions am preferably administered from about 1 to about 60 minutes prior to eating. More preferably, the compositions are administered within about 1 to about 30 minutes prior to eating. Most preferably, the compositions are administered from about 1 to about 5 minutes prior to eating.

The effective dosage unit for the compositions according to the invention may vary depending on the concentrations of nateglinide salt, the mode of administration, the condition being treated, and the severity of the condition being treated. Preferred dosage units contain an amount of a salt of nateglinide that corresponds to 40, 60, 120 and ISO mg of the free acid of nateglinide, respectively.

In addition, a variety of factors are specific to the patient being treated, such as species type, age, weight, and sex. In a preferred embodiment of the invention, the composition is administered to an adult patient in a dosage, corresponding to the free acid of nateglinide, in the range from about 50 to about 1200 mg/day, more preferably from about 90 to about 540 mg/day.

In one embodiment of the invention, the pharmaceutical compositions comprising at least one salt of nateglinide according to the invention are produced by a process that comprises granulating in the presence of water to form granules, drying the granules, and optionally screening the granules, for example, through a wire mesh screen. All of the ingredients of the composition may be added prior to or during the granulation. Alternatively, all or a portion of one or more of the ingredients may be added after the granulation step is complete. For example, all or a portion of anti-adherent (e.g., silica), all or a portion of lubricant (e.g., magnesium stearate) and/or all or a portion of disintegrant (e.g., croscarmellose or any salt thereof) may be added after the granulation.

The pharmaceutical compositions according to the invention may be used for preventing or treating diabetes, especially type 2 diabetes mellitus, cardiovascular diseases and conditions associated therewith. As used throughout the entire specification and in the claims the term, “cardiovascular diseases and conditions associated therewith” comprises hyperglycemia, hyperinsulinemia, hyperlipidaemia, insulin resistance, impaired glucose metabolism, obesity, diabetic retinopathy, macular degeneration, cataracts, diabetic nephropathy, glomerulosclerosis, diabetic neuropathy, erectile dysfunction, premenstrual syndrome, vascular restenosis, ulcerative colitis, coronary heart disease, hypertension, angina pectoris, myocardial infarction, stroke, skin and connective tissue disorders, foot ulcerations, metabolic acidosis, arthritis, osteoporosis, polycystic ovary syndrome (PCOS) and impaired glucose tolerance. As used herein, “preventing” means prophylactic administration of the composition to healthy patients or patients being in a pre-stage of diabetes to prevent the outbreak of the diseases and conditions mentioned herein.

The pharmaceutical compositions according to the invention may also be used for treating obesity by reducing the body weight of a patient. Hence, the invention relates also to a method of improving the bodily appearance of a mammal which comprises orally administering to said mammal a nateglinide salt composition as disclosed herein.

Another embodiment of the present invention relates to a method of treatment of diabetes, cardiovascular diseases, or conditions associated therewith. Such a method of treatment comprises the administration, to a mammal, especially a human, in need of such treatment, of an effective amount of a salt of nateglinide according to the invention or a combination thereof with other substances or compounds as described hereinbefore.

The following non-limiting examples illustrate further aspects of the invention.

### EXAMPLE 1

### Preparation of Sodium Salts of Nateglinide

### Compound 1

A solution of 23.81 g of nateglinide in 700 ml iso-propyl alcohol is stirred and 12.5 ml of 6 N sodium hydroxide solution in water are added. The mixture is stirred at room temperature for 1.5 to 2 hours. The resulting solids are isolated by suction filtration and washed with 50 ml isopropyl alcohol. The solids are then dried overnight at 55° C. and under vacuum (20 mm Hg). The water content of the obtained solid, as determined according to Karl Fischer, is 2.67%.

### Compound 2

A solution of 3.17 g of nateglinide in 35 ml of iso-propyl alcohol is stirred and treated by dropwise addition with 1.7 ml of 6 N sodium hydroxide. The mixture is stirred for one hour and after the solids appear, another 35 ml of iso-propyl alcohol is added at room temperature. The resulting solids are isolated by suction filtration and washed with IPA. The solids are dried at 55° C. under vacuum (20 mm Hg) overnight

### Compound 3

A stock solution of 1033 mg nateglinide in 50 ml ethanol yielding a solution with 0.0651 mmol nateglinide per ml ethanol is prepared.

10.678 mg sodium acetate are added to 2 ml of the above stock solution and stirred for 30 minutes at 40° C. The solution is evaporated to dryness and the solid residue is collected.

### Compound 4

A solution of 317.4 mg nateglinide in 1 ml ethanol is prepared. To this solution 1 ml of 1 N NaOH is added in two 0.5 ml portions. The precipitate is filtered off using whatman filter paper. The remaining solid is dried in a vacuum oven at 50° C., 27 mm Hg for 16 hours.

### EXAMPLE 2

### Preparation of Potassium Salts of Nateglinide

### Compound 5

A solution of 23.81 g of nateglinide in 700 ml of isopropyl alcohol is stirred and treated by dropwise addition with 12.6 ml of 6 N potassium hydroxide. The mixture is stirred at room temperature for one hour. The resulting solids are isolated by suction filtration and washed with 150 ml of 2:1 iso-propyl alcohol/ethyl acetate. The solids are dried at 55° C. under vacuum (20 mm Hg) overnight. The water content of the obtained solid, as determined according to Karl Fischer, is 2.12%.

### Compound 6

A solution of 3.17 g of nateglinide in 50 ml of iso-propyl alcohol is stirred and treated by dropwise addition with 2.0 ml of 5 N potassium hydroxide. The mixture is stirred at room temperature for one half hour. The resulting solids are isolated by suction filtration and washed twice with iso-propyl alcohol. The solids are dried at 55° C. under vacuum (20 mm Hg) overnight.

### Compound 7

For 24 hours Compound 5 is kept in a humidity chamber at 84% relative humidity resulting in Compound 7.

### Compound 8

To a solution of 309.17 mg nateglinide in 1.5 ml ethanol 1.5 ml 1N potassium hydroxide are added. The solution is stirred for 16 hours, the resulting slurry is then cooled to 4° C. and filtered using whatman paper filter. The remaining solid is dried in a vacuum oven at 50° C., 27 mm Hg, for 16 hours.

### EXAMPLE 3

### Preparation of Calcium Salts of Nateglinide

### Compound 9

A solution of 23.81 g of nateglinide in 1000 ml of deionized water is stirred and treated by dropwise addition with 75.0 ml of 1 N sodium hydroxide. The mixture is heated to 60-65° C. for 25 minutes to give a solution. The solution is cooled to 50° C. and filtered. A solution of 11.03 g of calcium chloride dihydrate in 100 ml of deionized water is added dropwise over one half hour. The resulting solids are isolated by suction filtration and washed with 250-300 ml of deionized water. The solids are dried at 55° C. under vacuum (20 mm Hg) overnight.

### Compound 10

A solution of 23.81 g of nateglinide in 700 ml of iso-propyl alcohol is stirred and treated by dropwise addition with 77.5 ml of 1 N sodium hydroxide. The mixture is heated to 55-60° C. for 15 minutes to give a solution. The solution is cooled to 25° C. and a solution of 6.06 g of calcium chloride dihydrate in 50 ml of water is added dropwise. After the addition, 250 ml of water are added and the slurry is stirred at room temperature for 18 hours. The resulting solids are isolated by suction filtration and washed with water. The solids are dried at 55° C. under vacuum (20 mm Hg) overnight. The water content of the obtained solid, as determined according to Karl Fischer, is 0.58%.

### EXAMPLE 4

### Preparation of Magnesium Salt of Nateglinide

### Compound 11

A solution of 3.17 g of nateglinide in 110 ml of deionized water and 4-5 ml of iso-propyl alcohol is stirred and treated by dropwise addition with 10.5 ml of 1 N sodium hydroxide. The mixture is heated to 80° C. to give a solution. The solution is cooled below 28° C. A solution of 2.03 g of magnesium chloride hexahydrate in 15 ml of deionized water is added dropwise. The resulting solids are isolated by suction filtration and washed with deionized water. The solids are dried at 55° C. under vacuum (20 mm Hg) overnight.

### EXAMPLE 5

### Preparation of N-Methyl-D-glucamine Salt of Nateglinide

A solution of 23.81 g of nateglinide in 350 ml of methanol is stirred and treated by dropwise addition with a solution of 14.79 g of N-methyl D-glucamine in 75 ml of 1:1 methanol/water. The mixture is stirred at room temperature for 35 minutes and then allowed to stand overnight. The resulting solids are isolated by suction filtration and washed with methanol. The solids are dried at 55° C. under vacuum (20 mm Hg) overnight. The water content of the obtained solid, as determined according to Karl Fischer, is <0.1%.

### EXAMPLE 6

### Preparation of Tris(hydroxymethyl)-aminomethane Salt of Nateglinide

A solution of 6.00 g nateglinide in 40 ml iso-propyl alcohol is prepared. To this solution 2.28 g of tris(hydroxymethyl)-aminomethane are added. The resulting solution is stirred for several hours at 40° C. Then the temperature is raised to 55° C. to evaporate excess solvent with stirring. The residue is dried by air flow, the remaining material is titurated with heptane and then filtered. The solids are dried by air flow.

### EXAMPLE 7

### Preparation of Lysine Salt of Nateglinide

### Compound 14

An amount of 6.00 g nateglinide is dissolved in 21 ml of iso-propyl alcohol. Thereto a solution of 2.76 g lysine in 12 ml water is added. The resulting solution is stirred for several minutes and placed in an ice bath. Stirring is continued until solid forms.

### Compound 15

A mixture of 3 ml acetone (93%; remainder: water) and 3 ml water is heated to 40° C. and 1.34 g nateglinide is dissolved therein. To this solution 0.62 g lysine are added and dissolved. Seed crystals are prepared by dissolving 0.53 g nateglinide in acetone (97%; remainder: water) and crushing by adding 015 g lysine. About 30 to 50 mg of the seeds are added to the solution. The gel-like material is dried by air flow.

### Compound 16

A stock solution of nateglinide is prepared by dissolving 1033 mg of nateglinide in 50 ml ethanol yielding a solution with 0.0651 mmol nateglinide per ml ethanol. To 5 ml of that stock solution 47.5 mg lysine are added and the slurry is stirred for several minutes at 40° C. to dissolve the lysine. Stirring is continued at the same temperature for 16 hours. The solution is cooled to 4° C. in a refrigerator for several weeks. Subsequently drying by air flow is affected. 5 ml iso-propyl alcohol are added to the remaining residue. Excess solvent is evaporated by air flow. The resulting solids are collected.

### EXAMPLE 8

### Preparation of Ammonium Salts of Nateglinide

### Compound 17

A solution of 23.81 g of nateglinide in 700 ml of iso-propyl alcohol is stirred and treated by dropwise addition with 10.5 ml of concentrated ammonium hydroxide. After 20 minute; 150 ml of ethyl acetate is added. The mixture is stirred at room temperature for 1.75 hours. The resulting solids are isolated by suction filtration and washed with 150 ml of 2:1 iso-propyl alcohol/ethyl acetate. The solids are dried at 55° C. under vacuum (20 mm Hg) overnight. The yield is 8.94 g. The water content, as determined according to Karl Fischer, is <0.1%.

### Compound 18

A solution of 3.17 g nateglinide in 50 ml iso-propyl alcohol is stirred and treated by dropwise addition with 1.4 ml of concentrated ammonium hydroxide. Then, 40 ml ethyl acetate are added. The mixture is stirred at room temperature for one hour. The resulting solids are isolated by suction filtration and washed with ethyl acetate. The solids are dried at 55° C. under vacuum (20 mm Hg) overnight.

### Compound 19

To a solution of 10.02 g nateglinide in 20 ml ethanol 1.1 ml concentrated ammonium hydroxide are added. The solution is stirred at 35° C. and 100 ml acetonitrile are added. The stirring is continued for several minutes. The solids are collected by filtration using whatman paper filter and further dried by air flow.

### EXAMPLE 9

### Pharmaceutical Composition Comprising the Salts of Nateglinide Prepared in Examples 1-8

### Composition

| intra-granular: | | |
|---|---|---|
| salt of nateglinide | 120 | mg |
| lactose monohydrate | 200-350 | mg |
| microcrystalline cellulose | 90-200 | mg |
| povidone | 10-30 | mg |
| croscarmellose sodium | 10-30 | mg |
| extra-granular: | | |
| magnesium stearate | 1-15 | mg |
| opadry white | 10-30 | mg |

Microcrystalline cellulose, povidone, croscarmellose sodium, the salt of nateglinide, e.g. the sodium salt of nateglinide, and lactose were mixed in a high shear mixer and afterwards granulated using purified water. The wet granules were dried in a fluid bed dryer and passed through a screen. Colloidal silicon dioxide was mixed, passed through a screen and blended with the dried granules in a V-blender. Magnesium stearate was passed through a screen, blended with the blend from the V-blender and afterwards the total mixture was compressed to tablets. Opadry yellow was suspended in purified water and the tablets were coated with the coating suspension.

The salts of nateglinide according to the invention have a high degree of dissociation in water, and thus, substantially improved water solubility. These properties are advantageous, since the dissolving process is quicker, and a smaller amount of water is required to prepare solutions containing such salts. In addition, the higher water solubility can, under certain conditions, lead to increased biological availability of the salts or salt hydrates in the case of solid dosage forms which is beneficial to patients

In the following the Tables I to IV selected analytical data are represented. Table IV represents selected characteristic peaks of reflection maxima in XRPD patterns of salts of nateglinide according to the present invention. In these tables compounds no. 1-4 are sodium salts of nateglinide, compounds 5-8 are potassium salts, compounds 9 and 10 are calcium salt; compound 11 is a magnesium salt, compound 12 is a N-methyl-D-glucamine salt, compound 13 is a tris(hydroxymethyl)-aminomethane salt, compounds 1416 are lysine salts, and compounds 17-19 are ammonium salts of nateglinide, respectively.

TABLE I
Evaluation of Nateglinide Salts

| | | LOH | MP | |
|---|---|---|---|---|
| Salt | Crystalline | DTA^{⋄} | DTA | Gain % |
| Free Acid | Crystalline | 0.0 | 140° C. | <0.1 |
| Form H | | | | |
| Sodium | Crystalline | 2.4% | 287° C. | 1.19 |
| (Compound No. 1) | | 124° C. | | |
| Sodium | Crystalline | 3.5% | 220° C. | — |
| (Compound No. 2) | | 78° C. | | |
| Sodium | Crystalline | 13.6% | 262° C. | — |
| (Compound No. 3) | | 55° C. | | |
| Sodium | Crystalline | 7.1% | 287° C. | — |
| (Compound No. 4) | | 96° C. | | |
| Potassium | Crystalline | 1.2% | 299° C. | 8.2* |
| (Compound No. 5) | | 144° C. | | |
| Potassium | Crystalline | 5.0% | 220° C. | — |
| (Compound No. 6) | | 61° C. | | |
| Potassium | Crystalline | 1.0% | 186° C. | — |
| (Compound No. 8) | | NA | | |
| Calcium | Crystalline/ | 5.7% | 282° C. | 0.4 |
| (Compound No. 9) | amorphous | 81° C. | | |
| Calcium | Crystalline/ | 4.9% | 250° C. | — |
| (Comp. No. 10) | amorphous | 97° C. | | |
| Magnesium | Crystalline | 4.9% | 268° C. | <0.1 |
| (Comp. No. 11) | | 92° C. | | |
| N-Methyl d- | Crystalline | 0.1% | 221° C. | <0.1 |
| glucamine | | | | |
| (Comp. No. 12) | | | | |
| TRIS | Crystalline | 2.1% | <60° C. | <0.1 |
| (Comp. No. 13) | | 60° C. | | |
| Lysine | Crystalline | 5.3% | 226° C. | 1.3 |
| (Comp. No. 14) | | 82° C. | | |
| Lysine | Crystalline | 2.0%* | 222° C. | — |
| (Comp. No. 15) | | NA | | |
| Lysine | Crystalline | 8.4% | 222° C. | — |
| (Comp. No. 16) | | 87° C. | | |
| Ammonium | Crystalline | 1.2%* | 123° C. | <0.1 |
| (Comp. No. 17) | | 123° C. | | |
| Ammonium | Crystalline | 4.0%* | 74° C. | — |
| (Comp. No. 18) | | 74° C. | | |
| Ammonium | Crystalline | 1.3%* | 154° C. | — |
| (Comp. No. 19) | | 146° C. | | |

— not measured
Crystallinity measured by X-Ray Powder Diffraction (XRPD)
LOH loss on heating
DTA Differential thermal analysis
Gain gain in weight at 84% RH (relative humidity)
*XRPD changed, confirmed by Thermalgravimetry

TABLE II
Elemental Analysis of Nateglinide Salts

| Salt | | | | | | KF—H₂O | |
|---|---|---|---|---|---|---|---|
| TRD | | Carbon | Hydrogen | Nitrogen | Metal | (%) | Comment |
| Free Base | Calc. | 71.88 | 8.59 | 4.41 | | | |
| Na⁺ | Calc. | 67.24 | 7.72 | 4.13 | Na, 6.77 | | |
| | Found | 67.02 | 7.84 | 4.04 | Na, 6.64 | 2.67 | Hemihydrate |
| K+ | Calc. | 64.19 | 7.37 | 3.94 | K, 10.97 | | |
| | Found | 63.62 | 7.29 | 3.84 | K, 10.58 | 2.12 | Hemihydrate |
| Ca⁺² | Calc. | 67.83 | 7.79 | 4.16 | ½Ca, | | |
| | | | | | 5.96 | | |
| | Found | 67.53 | 7.83 | 4.06 | Ca, 5.93 | | monohydrate |
| Mg⁺² | Calc. | 69.45 | 7.98 | 4.26 | ½Mg, | | |
| | | | | | 3.70 | | |
| | Found | 69.19 | 8.11 | 4.13 | ½Mg, | 5.20 | monohydrate |
| | | | | | 3.50 | | |
| NH₄+ | Calc. | 68.23 | 9.04 | 8.37 | — | | 1:1^{γ} |
| | | 70.00 | 8.83 | 6.45 | | | 2:1 |
| | | 70.61 | 8.75 | 5.78 | | | 3:1 |
| | | 70.93 | 8.74 | 5.44 | | | 4:1 |
| | Found | 70.90 | 8.94 | 5.58 | — | <0.10 | |
| | Found | 70.26 | 8.49 | 5.27 | — | 0.58 | |
| N-Me-D- | Calc. | 60.92 | 8.65 | 5.40 | — | | |
| Glucamine | | | | | | | |
| | Found | 60.82 | 8.46 | 5.43 | — | <0.10 | |
| “Tris” | Calc. | 62.99 | 8.73 | 6.39 | — | | Hydrate, variable |
| | Found | 63.73 | 8.69 | 6.33 | | 5.9 | |
| Lysine | Calc. | 64.75 | 8.93 | 9.06 | — | | Hydrate, variable |
| | Found | 62.2 | 9.02 | 8.38 | | 5.00 | |

^{γ}ratio nateglinide:NH₄

The data in Table II shows that theoretical and experimental values for the mineral, N-methyl glucamine and Tris(tris(hydroxymethyl)aminomethane) salts are in agreement. The data also shows that there are deviations with the ammonium and lysine salts.

The acid-base ratio of the ammonium salt has not been established. Elemental analysis indicates a possible 4:1 ratio; however, the solid is heated (−100° C.) to constant weight before analysis. Loss of some ammonium during the drying procedure is possible, however, significant loss would not occur until 110° C. (FIG. 10

TABLE III
Physical Data of Nateglinde Salts

| | | | Bulk | Solubility |
|---|---|---|---|---|
| | Acid:Base | Salt:Acid | Density | Water |
| Salt | Ratio | Ratio | g/cm³ | mg/ml |
| Sodium | 1:1 | 1.07 | 0.14 | >40 |
| Potassium | 1:1 | 1.12 | 0.17 | >40 |
| Calcium | 2:1 | 1.06 | 0.24 | 0.54 |
| Magnesium | 2:1 | 1.08 | 0.21 | 0.45 |
| Ammonium | 3:1 | 1.01 | 0.20 | <0.2 |
| N-methyl d- | 1:1 | 1.57 | 0.16 | 7.3 |
| glucamine | | | | |
| TRIS | 1:1 | 1.38 | 0.51 | >40 |
| Lysine | 1:1 | 1.46 | 0.50 | 7.1 |
| Form B of | | | 0.18 | 0.09 |
| nateglinide | | | | |
| Form H of | | | 0.28 | 0.09 |
| nateglinide | | | | |

TABLE IV

| | |
|---|---|
| | Position of selected |
| | maxima of reflection |
| Compound No. | in XRPD (2⊖) in degrees |

| | | | | |
|---|---|---|---|---|
| 1 | 4.5 | 5.1 | 16.3 | 18.4 |
| 2 | 3.4 | 4.5 | 4.9 | 5.4 |
| 3 | 5.0 | 8.8 | 17.9 | 29.8 |
| 4 | 4.6 | 13.8 | 17.0 | 18.3 |
| 5 | 4.8 | 5.4 | 15.1 | 15.9 |
| 6 | 4.9 | 5.0 | 19.9 | 20.0 |
| 7 | 4.4 | 4.9 | 13.3 | 16.2 |
| 8 | 4.7 | 5.5 | 13.5 | 15.4 |
| 9 | 4.8 | 15.5 | 18.6 | 19.3 |
| 10 | 4.3 | 5.1 | 18.4 | 18.7 |
| 11 | 4.2 | 5.7 | 13.5 | 19.9 |
| 12 | 7.8 | 11.2 | 12.9 | 20.4 |
| 13 | 16.7 | 18.2 | 20.0 | 21.7 |
| 14 | 8.6 | 18.3 | 18.8 | 20.3 |
| 15 | 8.4 | 19.2 | 20.2 | 23.8 |
| 16 | 4.3 | 7.4 | 10.6 | 14.9 |
| 17 | 4.7 | 4.8 | 13.7 | 15.4 |
| 18 | 4.7 | 13.4 | 15.3 | 18.4 |
| 19 | 5.2 | 13.1 | 19.4 | 21.3 |

In the following listing the positions of the reflection maxima (in degrees) together with their corresponding relative intensities are obtained in X-ray powder diffraction measurements are given for the Compounds 1 to 19.

| Compound 1 | | Compound 2 | | Compound 3 | |
|---|---|---|---|---|---|
| Position | Relative | Position | Relative | Position | Relative |
| (Deg.) | Intensity | (Deg.) | Intensity | (Deg.) | Intensity |
| 5.1 | 100.0 | 3.4 | 82.9 | 5.0 | 100.0 |
| 4.5 | 42.3 | 4.5 | 100.0 | 6.5 | 4.7 |
| 16.3 | 21.6 | 4.9 | 26.7 | 8.6 | 6.8 |
| 18.4 | 15.4 | 5.4 | 22.4 | 8.8 | 15.4 |
| 14.0 | 13.8 | 6.6 | 18.1 | 10.2 | 4.0 |
| 14.4 | 8.9 | 7.8 | 3.0 | 11.6 | 11.1 |
| 16.0 | 8.0 | 9.1 | 2.3 | 11.8 | 7.5 |
| 19.1 | 6.0 | 9.8 | 3.6 | 12.9 | 4.1 |
| 30.4 | 4.4 | 12.5 | 6.9 | 14.2 | 8.1 |
| 16.9 | 4.0 | 12.8 | 5.0 | 15.6 | 14.2 |
| 18.1 | 3.9 | 13.4 | 11.3 | 17.1 | 11.7 |
| 11.3 | 3.7 | 13.7 | 19.9 | 17.9 | 17.7 |
| 17.4 | 3.6 | 14.1 | 7.2 | 18.7 | 8.1 |
| 18.0 | 3.6 | 15.3 | 6.3 | 19.2 | 12.6 |
| 6.4 | 3.2 | 15.8 | 5.8 | 19.3 | 9.7 |
| 6.4 | 3.2 | 16.0 | 4.5 | 20.2 | 11.4 |
| 21.1 | 2.7 | 16.6 | 10.3 | 22.0 | 4.4 |
| 20.3 | 2.5 | 16.9 | 12.9 | 22.1 | 3.9 |
| 12.3 | 2.0 | 18.2 | 22.2 | 22.6 | 8.0 |
| 35.3 | 1.8 | 19.3 | 10.9 | 23.0 | 4.3 |
| 13.5 | 1.6 | 19.6 | 3.3 | 24.8 | 4.3 |
| 22.3 | 1.4 | 20.0 | 8.7 | 25.1 | 5.0 |
| 7.3 | 1.3 | 20.2 | 7.4 | 27.0 | 4.9 |
| 19.4 | 1.2 | 20.5 | 3.9 | 29.8 | 22.0 |
| 13.4 | 1.1 | 21.1 | 3.2 | 32.7 | 5.4 |
| 9.2 | 1.1 | 21.7 | 8.2 | 33.7 | 5.1 |
| 19.6 | 1.1 | 24.6 | 2.1 | 35.6 | 5.4 |
| 28.1 | 1.1 | 25.0 | 2.2 | 36.6 | 6.8 |
| 28.0 | 1.0 | 27.5 | 2.1 | 37.6 | 6.2 |
| 27.4 | 1.0 | 29.2 | 4.1 | | |
| 27.5 | 0.9 | 29.3 | 4.6 | | |
| 22.4 | 0.9 | 29.4 | 2.9 | | |
| Compound 4 | | Compound 5 | | Compound 6 | |
| Position | Relative | Position | Relative | Position | Relative |
| (Deg.) | Intensity | (Deg.) | Intensity | (Deg.) | Intensity |
| 4.6 | 100.0 | 4.8 | 100.0 | 4.9 | 100.0 |
| 5.4 | 14.0 | 5.4 | 28.1 | 5.0 | 96.6 |
| 6.7 | 14.3 | 13.8 | 12.4 | 5.4 | 4.7 |
| 7.9 | 3.1 | 15.1 | 34.4 | 6.8 | 13.7 |
| 9.3 | 2.5 | 15.9 | 28.4 | 14.4 | 9.1 |
| 12.7 | 8.6 | 18.4 | 7.4 | 14.6 | 5.3 |
| 13.0 | 6.2 | 18.9 | 17.1 | 14.9 | 14.8 |
| 13.5 | 6.8 | 21.7 | 4.2 | 15.0 | 12.4 |
| 13.8 | 16.0 | 23.1 | 3.2 | 15.2 | 11.4 |
| 14.3 | 9.3 | 23.2 | 3.5 | 16.0 | 10.2 |
| 15.0 | 2.8 | 23.3 | 3.4 | 17.0 | 7.0 |
| 15.5 | 1.6 | 24.9 | 3.5 | 17.4 | 7.5 |
| 15.6 | 1.6 | 26.4 | 4.2 | 18.6 | 23.4 |
| 15.9 | 4.8 | 30.4 | 4.1 | 18.8 | 23.1 |
| 17.0 | 16.6 | 30.5 | 3.1 | 19.1 | 23.5 |
| 17.3 | 2.4 | 30.8 | 3.1 | 19.4 | 17.2 |
| 17.4 | 3.2 | 30.9 | 3.7 | 19.6 | 11.0 |
| 18.3 | 19.0 | 31.1 | 4.0 | 19.9 | 32.7 |
| 19.4 | 14.0 | 31.3 | 2.9 | 20.0 | 33.3 |
| 19.7 | 2.1 | 34.9 | 3.3 | 20.4 | 12.7 |
| 20.2 | 5.9 | 35.0 | 3.8 | 20.5 | 11.8 |
| 20.7 | 1.9 | 35.2 | 2.9 | 20.6 | 10.8 |
| 20.8 | 2.8 | | | 20.9 | 4.7 |
| 21.8 | 7.1 | | | 21.0 | 7.5 |
| 22.8 | 2.2 | | | 21.1 | 6.9 |
| 23.0 | 1.9 | | | 21.4 | 6.7 |
| 23.9 | 1.9 | | | 22.3 | 11.5 |
| 23.9 | 2.7 | | | 22.4 | 12.6 |
| 24.7 | 2.8 | | | 22.5 | 10.2 |
| 25.1 | 2.1 | | | 25.3 | 5.6 |
| 27.7 | 2.0 | | | 25.5 | 6.9 |
| 29.4 | 5.8 | | | 28.6 | 8.0 |
| 33.6 | 2.3 | | | 28.9 | 7.3 |
| 33.7 | 2.7 | | | 29.0 | 4.5 |
| 35.2 | 2.0 | | | 37.9 | 4.2 |
| 37.9 | 2.0 | | | | |
| 38.2 | 1.8 | | | | |
| Compound 7 | | Compound 8 | | Compound 9 | |
| Position | Relative | Position | Relative | Position | Relative |
| (Deg.) | Intensity | (Deg.) | Intensity | (Deg.) | Intensity |
| 4.4 | 100.0 | 4.7 | 100.0 | 4.8 | 99.3 |
| 4.9 | 37.3 | 5.2 | 25.1 | 4.8 | 100.0 |
| 5.9 | 12.8 | 5.5 | 30.1 | 5.2 | 18.0 |
| 6.5 | 5.5 | 6.0 | 9.8 | 5.3 | 17.0 |
| 6.7 | 9.4 | 6.9 | 4.9 | 13.5 | 8.9 |
| 11.7 | 8.6 | 12.0 | 6.5 | 14.2 | 4.0 |
| 13.1 | 19.8 | 13.5 | 33.9 | 14.3 | 7.3 |
| 13.3 | 27.6 | 15.4 | 41.0 | 14.4 | 5.1 |
| 13.7 | 7.2 | 15.8 | 14.3 | 15.5 | 23.1 |
| 15.2 | 5.9 | 16.0 | 15.4 | 15.9 | 4.6 |
| 15.7 | 10.0 | 16.4 | 21.5 | 16.6 | 5.1 |
| 15.8 | 4.8 | 17.6 | 7.2 | 18.2 | 10.2 |
| 16.2 | 33.6 | 18.3 | 9.3 | 18.3 | 16.0 |
| 18.1 | 10.0 | 18.4 | 9.8 | 18.6 | 26.2 |
| 18.2 | 6.4 | 18.6 | 9.7 | 19.0 | 10.7 |
| 18.4 | 6.7 | 19.2 | 14.0 | 19.2 | 14.9 |
| 19.1 | 7.8 | 19.3 | 17.5 | 19.3 | 21.5 |
| 20.3 | 5.5 | 19.4 | 13.0 | 20.1 | 4.3 |
| 22.6 | 4.8 | 21.2 | 8.5 | 20.3 | 9.3 |
| 22.9 | 8.0 | 22.8 | 4.6 | 20.5 | 11.1 |
| 26.3 | 6.1 | 22.9 | 10.5 | 20.6 | 7.6 |
| | | 23.0 | 8.5 | 21.0 | 20.6 |
| | | 23.6 | 4.2 | 21.4 | 7.5 |
| | | 23.7 | 3.9 | 21.5 | 9.6 |
| | | 26.5 | 8.6 | 21.7 | 9.1 |
| | | 27.4 | 4.3 | 22.6 | 8.0 |
| | | 27.6 | 4.3 | 22.8 | 5.0 |
| | | 29.2 | 3.9 | 22.9 | 3.8 |
| | | 30.8 | 3.9 | 23.5 | 4.8 |
| | | 31.8 | 4.8 | 25.0 | 3.9 |
| | | 35.4 | 4.6 | 25.6 | 4.3 |
| | | | | 25.8 | 4.5 |
| | | | | 31.2 | 4.4 |
| Compound 10 | | Compound 11 | | Compound 12 | |
| Position | Relative | Position | Relative | Position | Relative |
| (Deg.) | Intensity | (Deg.) | Intensity | (Deg.) | Intensity |
| 4.3 | 24.1 | 4.2 | 53.2 | 7.8 | 100.0 |
| 5.1 | 100.0 | 5.7 | 100.0 | 8.3 | 16.9 |
| 6.4 | 9.4 | 7.4 | 3.0 | 9.0 | 5.6 |
| 6.7 | 6.4 | 7.4 | 3.5 | 11.2 | 53.3 |
| 9.4 | 4.9 | 7.5 | 3.6 | 12.9 | 47.9 |
| 9.9 | 2.3 | 8.1 | 2.0 | 15.7 | 29.4 |
| 10.5 | 2.4 | 10.7 | 5.7 | 16.4 | 38.0 |
| 12.3 | 3.6 | 11.0 | 2.3 | 17.9 | 31.6 |
| 13.5 | 2.7 | 13.5 | 28.1 | 18.3 | 11.6 |
| 13.7 | 2.6 | 13.5 | 28.3 | 18.6 | 5.5 |
| 14.6 | 6.0 | 15.4 | 8.2 | 19.2 | 25.0 |
| 15.1 | 3.0 | 15.9 | 11.8 | 19.7 | 5.9 |
| 17.2 | 8.3 | 16.0 | 12.7 | 20.4 | 51.4 |
| 17.4 | 5.9 | 16.1 | 10.3 | 22.5 | 7.8 |
| 17.6 | 6.0 | 16.7 | 3.2 | 23.1 | 11.6 |
| 18.2 | 11.0 | 17.1 | 9.8 | 24.1 | 13.6 |
| 18.4 | 11.6 | 18.6 | 7.2 | 25.7 | 6.6 |
| 18.7 | 18.0 | 18.8 | 9.7 | 27.6 | 6.7 |
| 19.3 | 3.1 | 18.9 | 9.6 | 27.8 | 6.3 |
| 19.6 | 7.5 | 19.3 | 3.0 | 29.9 | 6.7 |
| 20.6 | 6.5 | 19.4 | 1.9 | 32.2 | 5.1 |
| 20.9 | 5.5 | 19.7 | 14.9 | 33.2 | 9.0 |
| 21.9 | 9.1 | 19.9 | 17.6 | 35.0 | 8.8 |
| 22.3 | 3.8 | 20.1 | 13.6 | 38.8 | 4.8 |
| 22.4 | 4.3 | 20.6 | 2.0 | | |
| 22.5 | 4.9 | 20.7 | 3.0 | | |
| 23.5 | 3.5 | 20.9 | 2.7 | | |
| 24.4 | 3.0 | 21.0 | 3.0 | | |
| 24.9 | 3.3 | 21.3 | 4.7 | | |
| 25.1 | 3.1 | 21.5 | 3.1 | | |
| 25.2 | 3.4 | 22.3 | 4.8 | | |
| 25.4 | 3.9 | 22.4 | 5.6 | | |
| 25.5 | 3.9 | 22.6 | 6.3 | | |
| 27.7 | 4.6 | 22.7 | 5.3 | | |
| 31.2 | 2.4 | 22.8 | 7.3 | | |
| 36.7 | 2.0 | 23.0 | 5.8 | | |
| 39.2 | 2.0 | 23.2 | 4.4 | | |
| | | 23.6 | 1.8 | | |
| | | 26.7 | 2.6 | | |
| | | 32.3 | 1.7 | | |
| Compound 13 | | Compound 14 | | Compound 15 | |
| Position | Relative | Position | Relative | Position | Relative |
| (Deg.) | Intensity | (Deg.) | Intensity | (Deg.) | Intensity |
| 4.2 | 4.3 | 4.4 | 20.7 | 4.2 | 3.7 |
| 4.4 | 4.0 | 4.6 | 10.2 | 7.8 | 3.4 |
| 7.1 | 14.0 | 4.9 | 7.8 | 8.4 | 49.5 |
| 8.9 | 41.4 | 5.1 | 7.0 | 10.1 | 8.7 |
| 11.1 | 13.0 | 6.5 | 11.4 | 11.6 | 4.9 |
| 12.2 | 6.4 | 6.6 | 14.1 | 12.5 | 30.6 |
| 12.9 | 11.3 | 7.4 | 10.4 | 12.8 | 20.5 |
| 13.4 | 40.3 | 7.5 | 7.8 | 15.7 | 10.0 |
| 13.7 | 7.4 | 8.6 | 100.0 | 16.0 | 3.1 |
| 14.6 | 28.7 | 9.8 | 20.7 | 16.9 | 9.7 |
| 15.7 | 19.9 | 10.5 | 8.8 | 18.0 | 21.2 |
| 16.4 | 4.1 | 10.7 | 12.9 | 18.2 | 8.3 |
| 16.7 | 76.9 | 11.2 | 25.8 | 19.2 | 41.5 |
| 18.2 | 100.0 | 12.6 | 30.9 | 19.9 | 32.4 |
| 18.7 | 40.3 | 12.9 | 15.3 | 20.2 | 100.0 |
| 18.9 | 27.4 | 13.1 | 18.0 | 21.5 | 13.5 |
| 19.3 | 16.6 | 13.5 | 20.3 | 21.7 | 11.8 |
| 19.8 | 15.3 | 14.9 | 21.3 | 22.7 | 25.0 |
| 20.0 | 52.4 | 15.2 | 19.0 | 23.8 | 32.8 |
| 20.3 | 8.1 | 18.0 | 30.3 | 24.1 | 7.1 |
| 20.7 | 26.8 | 18.3 | 42.9 | 24.3 | 3.1 |
| 21.0 | 5.9 | 18.4 | 31.5 | 25.1 | 3.3 |
| 21.2 | 10.1 | 18.8 | 56.0 | 25.3 | 7.0 |
| 21.7 | 54.8 | 19.3 | 19.8 | 25.4 | 6.5 |
| 21.9 | 7.3 | 19.4 | 24.1 | 26.2 | 15.3 |
| 23.0 | 6.4 | 19.5 | 22.2 | 26.9 | 3.4 |
| 23.7 | 7.2 | 20.3 | 33.9 | 28.3 | 3.6 |
| 24.1 | 8.5 | 20.5 | 11.9 | 28.4 | 6.6 |
| 25.0 | 9.4 | 21.1 | 31.2 | 28.8 | 3.5 |
| 26.4 | 6.9 | 21.3 | 30.7 | 29.0 | 6.1 |
| 26.6 | 11.9 | 21.5 | 14.7 | 29.1 | 5.9 |
| 27.1 | 10.3 | 21.6 | 15.2 | 29.2 | 6.2 |
| 27.5 | 6.7 | 21.7 | 13.2 | 30.5 | 4.4 |
| 28.6 | 17.4 | 22.4 | 7.6 | 31.9 | 3.2 |
| 30.8 | 4.9 | 22.8 | 7.0 | | |
| 31.3 | 5.0 | 22.9 | 9.0 | | |
| 32.0 | 6.0 | 23.5 | 9.6 | | |
| 32.4 | 4.2 | 23.6 | 10.6 | | |
| 33.5 | 4.3 | 23.8 | 27.2 | | |
| 34.6 | 5.6 | 24.1 | 18.2 | | |
| 35.4 | 5.8 | 24.2 | 6.5 | | |
| 36.9 | 5.3 | 25.2 | 13.7 | | |
| | | 25.5 | 10.2 | | |
| | | 26.3 | 21.1 | | |
| | | 26.6 | 8.8 | | |
| | | 27.0 | 10.8 | | |
| | | 30.6 | 7.6 | | |

| | |
|---|---|
| Compound 16 | Compound 17 |

| Position | Relative | Position | Relative |
|---|---|---|---|
| (Deg.) | Intensity | (Deg.) | Intensity |
| 4.3 | 73.6 | 4.7 | 100.0 |
| 5.0 | 47.9 | 4.8 | 93.1 |
| 7.4 | 100.0 | 5.4 | 35.9 |
| 8.1 | 21.5 | 7.7 | 11.7 |
| 9.1 | 28.5 | 8.6 | 5.3 |
| 10.2 | 23.9 | 8.7 | 5.1 |
| 10.6 | 50.8 | 9.5 | 1.7 |
| 13.0 | 42.5 | 9.7 | 2.3 |
| 14.9 | 69.6 | 9.8 | 2.4 |
| 15.3 | 7.0 | 10.7 | 5.5 |
| 16.2 | 7.4 | 11.6 | 4.4 |
| 17.3 | 11.0 | 13.7 | 37.1 |
| 17.8 | 6.6 | 14.5 | 1.6 |
| 18.6 | 30.2 | 15.4 | 43.7 |
| 18.8 | 27.6 | 16.1 | 29.0 |
| 19.2 | 36.6 | 16.7 | 6.7 |
| 20.0 | 34.4 | 17.1 | 1.6 |
| 20.0 | 34.2 | 18.9 | 35.4 |
| 20.2 | 32.5 | 19.2 | 12.8 |
| 21.2 | 29.1 | 19.4 | 12.4 |
| 21.3 | 27.5 | 20.3 | 2.3 |
| 21.4 | 25.6 | 20.6 | 2.5 |
| 21.7 | 23.7 | 21.4 | 8.1 |
| 22.7 | 17.8 | 21.8 | 1.6 |
| 22.9 | 9.4 | 22.9 | 1.5 |
| 23.0 | 6.6 | 23.2 | 2.7 |
| 24.2 | 8.4 | 23.2 | 2.7 |
| 24.9 | 11.5 | 27.5 | 1.7 |
| 25.0 | 12.4 | 27.8 | 1.4 |
| 25.1 | 15.6 | 29.7 | 1.3 |
| 25.3 | 18.6 | 29.9 | 1.8 |
| 25.4 | 9.0 | 30.1 | 1.5 |
| 26.4 | 13.6 | | |
| 26.5 | 12.3 | | |
| 27.4 | 10.1 | | |
| 27.5 | 14.3 | | |
| 28.6 | 8.3 | | |
| 28.7 | 9.2 | | |
| 31.6 | 7.0 | | |

| | |
|---|---|
| Compound 18 | Compound 19 |

| Position | Relative | Position | Relative |
|---|---|---|---|
| (Deg.) | Intensity | (Deg.) | Intensity |
| 4.7 | 100.0 | 5.2 | 100.0 |
| 5.2 | 11.9 | 9.8 | 7.8 |
| 7.7 | 8.9 | 13.1 | 76.4 |
| 8.6 | 6.1 | 15.1 | 5.8 |
| 10.3 | 7.4 | 18.3 | 12.7 |
| 11.7 | 1.8 | 19.4 | 41.9 |
| 11.8 | 2.1 | 20.1 | 28.7 |
| 13.4 | 17.6 | 21.3 | 29.6 |
| 15.3 | 64.2 | 21.7 | 3.9 |
| 16.0 | 10.7 | 22.1 | 3.0 |
| 18.4 | 29.7 | 22.4 | 9.3 |
| 19.3 | 5.3 | 23.6 | 24.2 |
| 20.5 | 6.7 | 24.7 | 7.5 |
| 20.7 | 4.0 | 25.3 | 3.0 |
| 21.5 | 3.7 | 26.6 | 9.2 |
| 22.9 | 2.1 | 29.2 | 5.4 |
| 23.0 | 2.9 | 30.3 | 8.0 |
| 23.2 | 3.8 | 31.0 | 3.2 |
| 26.0 | 2.2 | 31.1 | 3.9 |
| 30.4 | 1.7 | 31.5 | 7.6 |
| 30.6 | 1.7 | 34.6 | 3.4 |
| 30.8 | 1.8 | | |
| 31.2 | 3.9 | | |
| 32.7 | 1.9 | | |
| 37.2 | 2.0 | | |

1. A salt of nateglinide having a melting point in the range of 50 to 300° C.

2. A salt of nateglinide according to claim 1 having a melting point in the range of 50 to 125° C.

3. A salt of nateglinide according to claim 1 having a melting point in the range of 150 to 300° C.

4. A salt of nateglinide having a solubility in water of at least 0.18 mg/ml.

5. A salt of nateglinide according to claim 4 having a solubility in water of at least 0.4 mg/ml.

6. A salt of nateglinide according to claim 5 having a solubility in water of at least 40 mg/ml.

7. A salt of nateglinide according to claim 1 having an X-ray powder diffraction (XRPD) pattern that comprises a combination of reflection maxima as set forth in Table IV.

8. A salt of nateglinide according to claim 7, which is present in an amorphous form.

9. A salt of nateglinide according to claim 7, which is present in a crystalline form.

10. A salt of nateglinide according to claim 7, which is present as a mixture of an amorphous form and a crystalline form.

11. A salt of nateglinide according to claim 1, in which the cation is selected from the group consisting of Na⁺, K⁺, Ca²⁺, Mg²⁺, the protonated form of Tris(hydroxymethyl)-aminomethane, the protonated form of N-methyl-D-glucamine and a protonated form of Lysine.

12. A salt of nateglinide according to claim 11, in which the ratio of the nateglinide anion to the cation is 1:1.

13. A salt of nateglinide according to claim 11, in which the ratio of the nateglinide anion to the cation is 2:1.

14. A salt of nateglinide according to claim 1 that loses between 0.1 and 14% of its mass on heating.

15. A salt of nateglinide according to claim 14 that loses between 0.1 and 9% of its mass on heating.

16. A salt of nateglinide according to claim 1 having a bulk density between 0.1 and 0.6 g/cm³.

17. A composition comprising a salt of nateglinide according to claim 1.

18. A composition according to claim 17 comprising one or more additional ingredients selected from the group consisting of vitamins, nutrition supplements and pharmaceutically active substances.

19. A composition according to claim 18 comprising nateglinide or repaglinide as additional ingredient.

20. A composition according to claim 18 wherein the pharmaceutically active substance is selected from the group consisting of insulin sensitizers, insulin secretion enhancers, Dipeptidyl peptidase IV inhibitors, ACE inhibitors and angiotensin 11 inhibitors.

21. A composition according to claim 18, which is a combined preparation or pharmaceutical composition.

22. A pharmaceutical composition according to claim 21 for the treatment of diabetes, cardiovascular diseases, or conditions associated therewith.

23. A method of treatment of diabetes, cardiovascular diseases, or conditions associated therewith comprising the administration, to a mammal in need of such treatment, of an effective amount a salt of nateglinide according to claim 1.

24. A method of treatment according to claim 23 wherein the cardiovascular diseases or conditions associated therewith are selected from the group consisting of hyperglycemia, hyperinsulinemia, hyperlipidaemia, insulin resistance, impaired glucose metabolism, obesity, diabetic retinopathy, macular degeneration, cataracts, diabetic nephropathy, glomerulosclerosis, diabetic neuropathy, erectile dysfunction, premenstrual syndrome, vascular restenosis, ulcerative colitis, coronary heart disease, hypertension, angina pectoris, myocardial infarction, stroke, skin and connective tissue disorders, foot ulcerations, metabolic acidosis, arthritis, osteoporosis, polycystic ovary syndrome (PCOS) and impaired glucose tolerance.
